(19) United States
(12) Patent Application Publication
(10) Pub. No.: US 2022/0323523 A1
(43) Pub. Date: Oct. 13, 2022

(54) METHOD AND APPARATUS FOR THE INFUSION OF CANNABINOIDS INTO A SHELF-STABLE FOOD COMPOSITION

(71) Applicant: Gianna Marie FILIPETTO, Aledo, TX (US)

(72) Inventor: Gianna Marie FILIPETTO, Aledo, TX (US)

(21) Appl. No.: 17/638,806

(22) PCT Filed: Aug. 25, 2020
(86) PCT No.: PCT/US2020/047788

§ 371 Date: Feb. 26, 2022

Related U.S. Application Data
(60) Provisional application No. 62/891,742, filed on Aug. 26, 2019.

(51) Int. Cl. A61K 36/185; A61K 9/00; A61K 47/44; A23L 33/12; A23L 33/105; A23D 9/013

(52) U.S. Cl. CPC A61K 36/185; A61K 9/0053; A61K 47/44; A23L 33/12; A23L 33/105; A23D 9/013; A23V 2002/00

(57) ABSTRACT

The present invention is a method and apparatus for the infusion of cannabinoids into a shelf-stable food composition. The composition contains a combination of two or more lipid-based oils with the addition of an emulsifier. The combination of oils presents a composition rich in varied length triglycerides which aids in the process of infusing lipophilic plant compounds (e.g. cannabinoids) into lipid-based foods. The unique varied triglyceride makeup of the present invention increases bioavailability of cannabinoids upon consumption. The core components of the invention are an oil rich in fat such as coconut oil, clarified butter, and an emulsifying agent such as lecithin, which, generally speaking, are configured as follows: an oil rich in saturated fats such as coconut oil is combined with a second oil rich in saturated fats such as clarified butter, and an emulsifying agent such as lecithin. The components are mixed until well blended.

### BACKGROUND

### Field of the Invention

The invention relates generally to method and apparatus for the infusion of cannabinoids into a shelf-stable food composition.

### Background

Currently there are a number of solutions for infusing food with cannabinoids. The most widely practiced of these solutions is the process of infusing butter with the cannabis or hemp plant. This process takes place by combining water, butter, and decarboxylated lipophilic herbal ingredients in a saucepan and placing it on the stove. This process takes well over three hours for the full infusion to take place on the stovetop. Once the infusion is complete the user strains the butter and places it in a bowl in the refrigerator while it hardens. The hardening time can take up to 6 hours depending on the amount of herbal butter the user makes. Once the butter hardens, it separates from the water. The user then has to separate the water from the hardened butter by placing a small incision on the side of the bowl to drain the water out. This method requires a significant amount of time for the herbal infusion to take place and often has inconsistent outcomes. A significant portion of cannabinoids will be lost in the process due to the long simmering time as well as having only one lipid-based carrier for the lipophilic plant compounds to bind to. Some solutions seek to increase the potency of the herbal butter by placing it in a crock-pot for an extended period of time, usually 8 hours. These methods are unable to produce herbal butter that will increase cannabinoid bioavailability due to the lack of lipid based compounds in butter and result in the loss of beneficial cannabinoids.

### SUMMARY OF THE INVENTION

It is desirable to have a shelf-stable composition that readily absorbs cannabinoids in a straightforward infusion process. Furthermore, it is desirable to have a composition that dramatically reduces the time it takes to make lipid-based herbal infusions when compared to traditional infusion methods. Still further, it is desirable to have a composition that has an outcome in which increased bioavailability of cannabinoids are offered when the composition is administered orally or added as an oil to other foods for consumption. The disclosed device advantageously fills these needs and addresses the aforementioned deficiencies by providing a shelf-stable composition that dramatically reduces the time it takes to make herbal infusions while offering a specially-formulated outcome that increases the bioavailability of beneficial plant compounds during the digestion process.

Disclosed is a method and apparatus for the infusion of lipophilic plant compounds into food, which is made up of the following components: (1) coconut oil (2) Clarified butter or ghee to help facilitate transportation to the target organ and final delivery inside the cell via the cell membrane (3) lecithin can be added to act as an emulsifying agent. Parts (1), (2), and (3) are combined and evenly blended together to produce a specially formulated composition that aids in the transportation of beneficial plant compounds to the user. The user adds the present invention to a pan and places it on a stovetop, adding lipophilic herbal ingredients to the pan. The user then places the stovetop on a low heat for approximately 15-20 minutes or until the invention has enough time to bind to the hydrophobic plant material. Once the infusion has occurred, the invention can be removed from the stovetop where the plant material can be strained out. Upon removing the plant material, the user has an oil rich in cannabinoids that can be added as an ingredient to other foods or be orally administered.

The device may also have one or more of the following: an additional oil may be added as a third oil or used in place of coconut oil or clarified butter such as: safflower oil canola oil, flaxseed oil, sunflower oil, margarine, butter, corn oil, olive oil, sesame oil, soybean oil, peanut oil, cottonseed oil, vegetable shortening, chicken fat, lard, beef tallow, palm oil, cocoa butter, palm kernel oil, rapeseed oil, Medium-chain triglycerides oil (MCT oil), avocado oil, hemp oil, milk fat, palm oil, grapeseed oil, mustard oil, rice bran oil, almond oil. Flavor additives and/or herbal ingredients may be added to the invention. Lecithin may be removed from the invention or another emulsifying agent may be used in addition to or in its place such as agar, albumin, alginates, casein, egg yolk, glycerol monostearate, gums, Irish moss, soaps.

The disclosed device is unique when compared with other known devices and solutions because it provides: (1) a pre-made composition that dramatically reduces the time it takes for herbal infusions to take place. Traditional infusion methods take a minimum of three hours of simmering herbal butter on a low setting for the infusion to take place. The present invention reduces the simmering time to 15-20 minutes for the infusion process to be complete. Due to the uniquely high fat content makeup of the present invention the infusion process is complete significantly faster than other known solutions (2) an herbal infusion that increases the bioavailability of cannabinoids upon consumption. One study found that the co-administration of cannabinoids with lipids enhanced the bioavailability of THC in rats by more than 2.5-fold and of CBD by almost 3-fold. The present inventions varied lengths of fatty acids requires the herbal infusion to be processed by the body through two different systems therefore increasing the bioavailability of beneficial plant compounds in the body. (3) a shelf-stable product users can purchase to complete their herbal infusions at home. The present invention requires far less steps than other known solutions making it more efficient for users to perform herbal infusions independently.

The disclosed device is unique in that it is structurally different from other known devices or solutions. More specifically, the device is unique due to the presence of: (1) a uniquely high saturated fat content makeup allowing for beneficial lipophilic plant compounds to more readily bind to lipids found in the present invention; (2) a variety of short-chain fatty acids, medium-chain fatty acids, and long-chain fatty acids increasing the bioavailability of cannabinoids in the body upon consumption due to the separate routes these acids take during the digestion process; (3) the use of a combination of oils in an herbal infusion. The present invention is the first to use more than one lipid based oil to perform an herbal infusion; (4) the use of an emulsifying agent such as lecithin to act as both a surfactant as well as bind to food the oil can later be added to more efficiently.

This disclosure will now provide a more detailed and specific description that will refer to the accompanying drawings. The drawings and specific descriptions of the drawings, as well as any specific or alternative embodiments discussed, are intended to be read in conjunction with the entirety of this disclosure. The Method and apparatus for the infusion of cannabinoids into a shelf-stable food composition may, however, be embodied in many different forms and should not be construed as being limited to the embodiments set forth herein; rather, these embodiments are provided by way of illustration only and so that this disclosure will be thorough, complete and fully convey understanding to those skilled in the art.

### BRIEF DESCRIPTION OF THE DRAWINGS

FIG. 1. is a flowchart illustrating how the shelf-stable composition functions.

FIG. 2. is an illustration depicting how the shelf-stable composition is blended and packaged.

FIG. 3. depicts the shelf-stable composition and ground herb being placed in a pan on a stove top and placed on a low heat.

FIG. 4. illustrates how hydrophobic plant compounds such as Tetrahydrocannabinol and Cannabidiol bind to fatty acids such as Oleic Acid and Linoleic Acid.

FIG. 5. shows how the shelf-stable composition and ground herb mixture is strained to separate the plant material from the completed herbal infusion.

FIG. 6. is a diagram illustrating how the LCFAs contained within the shelf-stable composition are processed through the human small intestine to the lymphatic system.

FIG. 7. is a diagram illustrating how the SCFAs and MCFAs contained within the present invention are processed through the human Small Intestine ultimately to the hepatic portal vein.

FIG. 8. is a diagram illustrating the two routes the various length fatty acids contained within the present invention are processed through the human digestive system.

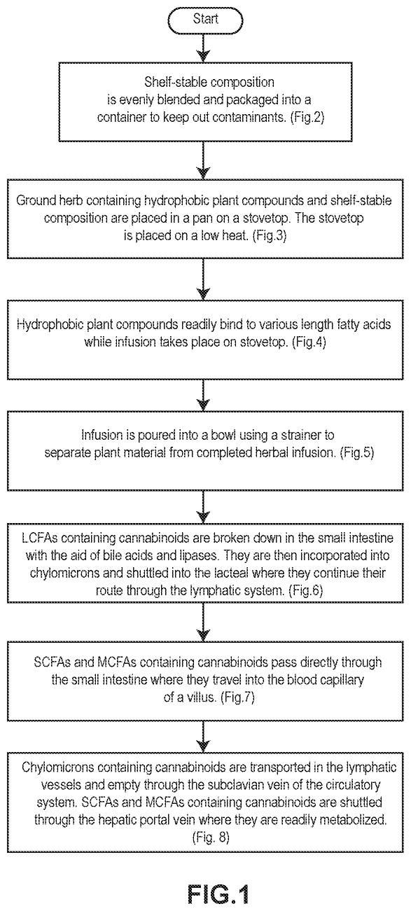

### DETAILED DESCRIPTION

The present invention is directed to method and apparatus for the infusion of cannabinoids into a shelf-stable food composition.

In its most complete version, the device is made up of the following components: (1) coconut oil (2) clarified butter and (3) lecithin to act as an emulsifying agent. Parts (1), (2), (3), are mixed together until evenly blended throughout.

Coconut oil is approximately 90% saturated fat and is made up primarily of medium-chain fatty acids. Coconut oil is an important component to the present invention due to its high saturated fat content makeup. Research has shown that cannabinoids are lipophilic and will therefore bind more readily to the saturated fat found in coconut oil during the infusion process. Upon consumption of the present invention, the medium-chain fatty acids are broken down and metabolized more readily than comparable lipid-based oils composed of long-chain fatty acids. The medium-chain fatty acids found within coconut oil travel directly through the small intestine and route through the hepatic portal vein. Coconut oil is unique in its makeup of high saturated fat without the damaging effects of long-chain fatty acids.

Clarified butter is a valuable source of lipophilic vitamins A, D, E, and K. Clarified butter is composed of long-chain fatty acids as well as short-chain fatty acids. This mixture of fatty acids found within the present invention helps increase the bioavailability of cannabinoids upon consumption due to the difference in how the body digests triglycerides of varied lengths. Research suggests that approximately 95% of the fat is available for digestion when the mixture of fatty acids is varied. Short and medium-chain fatty acids such as those found in coconut oil are broken off from the triglycerides without the need for bile acids. They are then transported directly to the liver through the portal artery without the use of chylomicrons. In comparison the digestion of long-chain triglycerides, such as those found within clarified butter, start in the small intestine and is completed with the aid of bile acids and lipases. The lipases break the triglycerides into individual fatty acids and monoglycerides in the small intestine while the bile acids allow the triglycerides to be properly emulsified.

When these parts are absorbed through the wall of the intestine, they are reassembled into triglycerides and carried into the body through the lymph system on chylomicrons. The digestion of short- and medium-chain fatty acids does not require help from bile acids and monoglycerides to be metabolized. The present invention's combination of long-chain fatty acids, short-chain fatty acids, and medium-chain triglycerides increases the bioavailability of cannabinoids due to the two different systems that the varied length fatty acids are metabolized through. Coconut oil and clarified butter are ideal for infusions due to their high boiling points. The present invention increases the bioavailability of cannabinoids in the body without a high concentration of long-chain fatty acids, which have been shown to have damaging effects on the heart and cardiovascular system.

The best combination of lipid-based oils is coconut oil and clarified butter however, the present invention will work if an additional oil is added such as: safflower oil, canola oil, flaxseed oil, sunflower oil, margarine, butter, corn oil, olive oil, sesame oil, soybean oil, peanut oil, cottonseed oil, vegetable shortening, chicken fat, lard, beef tallow, palm oil, cocoa butter, palm kernel oil, rapeseed oil, medium-chain triglyceride oil, avocado oil, hemp oil, milk fat, palm oil, grapeseed oil, mustard oil, rice bran oil, or almond oil.

Lecithin is used as an emulsifying agent to enable ingredients to bind together more readily. By adding lecithin to the oil and butter mixture, the final outcome of the infusion will result in an oil that is best when added as an oil component to other foods. Furthermore, lecithin is known to be a surfactant, meaning it can help to distribute the herbal ingredients such as cannabinoids more efficiently throughout. Liquid lecithin is the best form to use due to its ability to evenly blend with the coconut oil and clarified butter. Lecithin derived from sunflower seeds, eggs, and soybeans will be most commonly used for this invention but emulsifying agents such as agar, albumin, alginates, casein, egg yolk, glycerol monostearate, gums, Irish moss, soaps can be used in its place if necessary.

The present invention is added to a pan and placed on a stovetop. Next, the user will add finely ground decarboxylated lipophilic herbal ingredients and place the stove on a low heat, stirring occasionally. The oily composition should never come to a boil to preserve the natural plant compounds. In approximately 15-20 minutes the infusion process will be complete. The oily composition will turn a darker shade and is ready to be strained. The present invention is packaged and sold in a container to keep outside contaminants away. The present invention can be stored at room temperature due to the shelf-stable nature of the composition.

Research has shown when a person eats a cannabis infused food containing a high-fat ingredient, these fats appear to stimulate activity in the intestinal lymphatic system. In this way, the compounds that are dissolved in fats are transported into the bloodstream by this secondary lymphatic system and made available to the body. One study found that the co-administration of lipids enhanced exposure of rats to cannabinoids, THC by 2.5-fold and CBD by 3-fold when compared to lipid-free formulations. The present invention is the first shelf-stable oil composition that has been specially formulated for the infusion of cannabinoids into food.

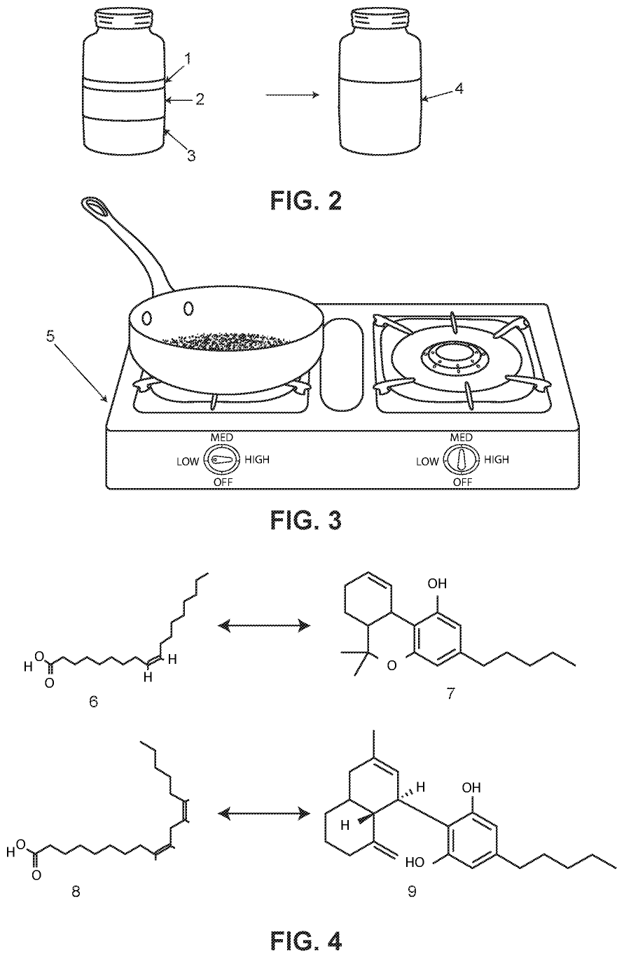

FIG. 2 shows an illustration of how the shelf-stable composition may be assembled and packaged. In this illustration, Lecithin (1) is blended with Coconut Oil (2) and Clarified Butter (3) resulting in the blended composition (4). These components are the preferred ingredients due to their varied length fatty acid makeup and shelf-stable elements. Liquid Lecithin (1) is a preferred emulsifying agent due to its ability to blend readily with the other ingredients. Lecithin (1) derived from sunflower seeds, eggs, and soybeans will be most commonly used for this invention. Emulsifying agents such as agar, albumin, alginates, casein, egg yolk, glycerol monostearate, gums, Irish moss, or soaps can be used in its place if necessary. The emulsifying agent may be removed for the invention to work however it is highly recommended. It is preferred that approximately 1 tablespoon of Lecithin (1) is used per cup of the remaining ingredients. Although the ratio of 1 tablespoon of Lecithin (1) to 1 cup of oil is preferred, more or less lecithin may be added depending on the preference of the user.

In FIG. 2 an equal amount of Coconut Oil (2) and Clarified Butter (3) is used. The illustration depicts ½ cup of Coconut Oil (2) and ½ cup of Clarified Butter (3) with 1 tablespoon of Lecithin (1). This depiction is the preferred ratio however the user can increase or decrease the ratio of Coconut Oil (2) to their liking. The user can also increase or decrease the ratio of Clarified Butter (3) to their liking.

Coconut Oil (2) and Clarified Butter (3) are the preferred oils due to their unique saturated fat content makeup however more oils may be used in addition or in place of them such as safflower oil, canola oil, flaxseed oil, sunflower oil, margarine, butter, corn oil, olive oil, sesame oil, soybean oil, peanut oil, cottonseed oil, vegetable shortening, chicken fat, lard, beef tallow, palm oil, cocoa butter, palm kernel oil, rapeseed oil, medium-chain triglyceride oil, avocado oil, hemp oil, milk fat, palm oil, grapeseed oil, mustard oil, rice bran oil, almond oil. Flavor additives may be added as well as additional herbal ingredients.

Once the preferred ingredients are added they are evenly blended and may be packaged in an airtight container to keep contaminants out. The blended composition (4) may be kept at room temperature or in a refrigerator. The present invention may be manufactured and sold to consumers in a similar airtight container at room temperature.

FIG. 3 illustrates the first infusion step for the shelf-stable composition. The user takes the blended composition (4) and adds it to a pan with hydrophobic decarboxylated ground herb. It is preferred to use approximately ½ of an ounce of ground herbal ingredients per approximately ½ cup of the composition (4). The user may use as little or as much ground herb as they prefer with more herb making the infusion more potent. Once the user adds their decarboxylated ground herb and the composition (4) to a pan on the stovetop, they place the stove on a low heat (5). The composition is to be left on the stove (5) for 15-20 minutes while the infusion takes place. The user can allow the herbal infusion to simmer for as long as they desire. During this time the composition will darken in color. The user should stir the composition occasionally and make sure that it never comes to a boil to preserve beneficial plant compounds.

FIG. 4 illustrates the binding of hydrophobic plant compounds with fatty acids contained within the composition (4) that occurs during the infusion process in FIG. 3.

The chemical structure of Oleic Acid (6) is used as an example of a fatty acid that is present in Coconut Oil. The arrow demonstrates Oleic Acid (6) binding to hydrophobic plant compound Tetrahydrocannabinol (7) found in the cannabis plant during the infusion process. Oleic Acid (6) is purely used as an illustrative example of a fatty acid found within the composition (4). Other examples of fatty acids that may be found within the composition are Laurie acid, Caprylic acid, Decanoic acid, Myristic acid, and Pathmitic Acid. While the herbal infusion is taking place on the stove (5) the above fatty acids may readily bind with the decarboxylated ground herb.

The chemical structure of Linoleic Acid (8) is used as an illustrative example of a fatty acid that is typically found within Clarified Butter. The arrows pointing to Linoleic Acid (8) and Cannabidiol (9) represent the chemical binding that takes place in FIG. 3 during the stovetop infusion process. Linoleic Acid (8) is used as an example of one fatty acid found in Clarified Butter. Cannabidiol (9) and Tetrahydrocannabinol (7) are illustrative examples of Cannabinoids that may be found within the decarboxylated herb that is added to the present invention. To date there are 113 Cannabinoids that have been isolated, most of which are hydrophobic. Cannabidiol (9) and Tetrahydrocannabinol (7) are just two examples of the Cannabinoids that will bind to the fatty acids found within the composition (4).

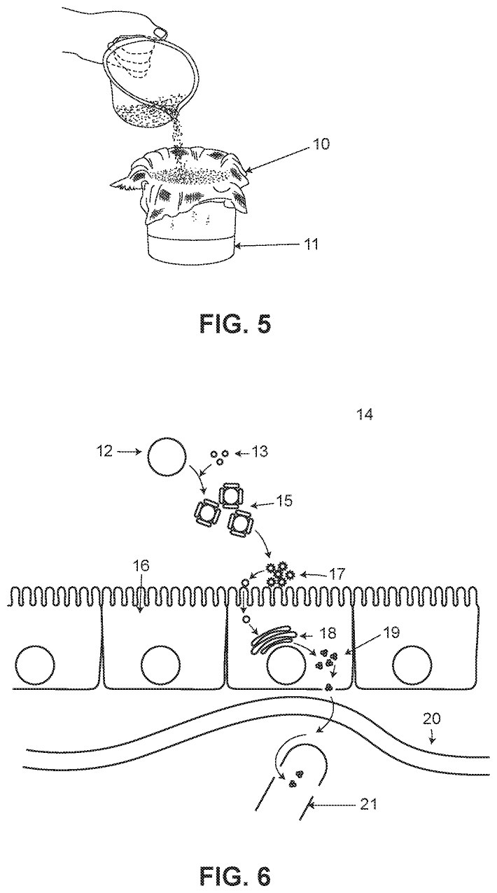

FIG. 5 is an illustration that depicts the step that occurs after FIG. 3 when the herbal infusion is complete. Once the herbal infusion on the stovetop (5) is done simmering the user may strain out the ground herb using a strainer cloth (10). The strainer cloth (10) is used to capture the ground herb as the completed herbal infusion (11) is poured into a bowl. A strainer cloth (10) is an illustrative example of how the user will separate their ground herb from the completed infusion (11). The strainer cloth (10) may be made of cheesecloth or another type of strainer may be used such as those made of a metal or mesh material. Once the straining step is complete the user is left with the completed herbal infusion (11). The herbal infusion (11) is ready for consumption by either being eaten directly or added to other foods. Typically the completed herbal infusion (11) will be added to other foods with high fat content to increase the bioavailability of the hydrophobic plant compounds found within the infusion. The herbal infusion (11) is preferably added in place of oils such as vegetable oil, butter, coconut oil, and olive oil, into a wide array of recipes.

FIG. 6 is an illustration that depicts a long-chain fatty acid fat globule containing cannabinoids (12) as it is metabolized by the small intestine. Due to the unique fat makeup of the present invention, the bioavailability of cannabinoids is increased because of the separate ways varied length fatty acids are metabolized. FIG. 6 illustrates how long-chain fatty acids found within the present invention are processed upon consumption through the human body. When a fat globule containing cannabinoids (12) is digested it is aided by bile salts (13) and emulsified (15) as the fat globule is broken down into smaller pieces in the lumen of the intestine (14). Fatty acids leave micelles (17) and enter the epithelial cells of the small intestine (16). Once inside the small intestine fat globules containing cannabinoids (12) combine with proteins to form chylomicrons (19) inside the golgi apparatus (18). Chylomicrons (19) exit the epithelial cell where they enter a lacteal (21).

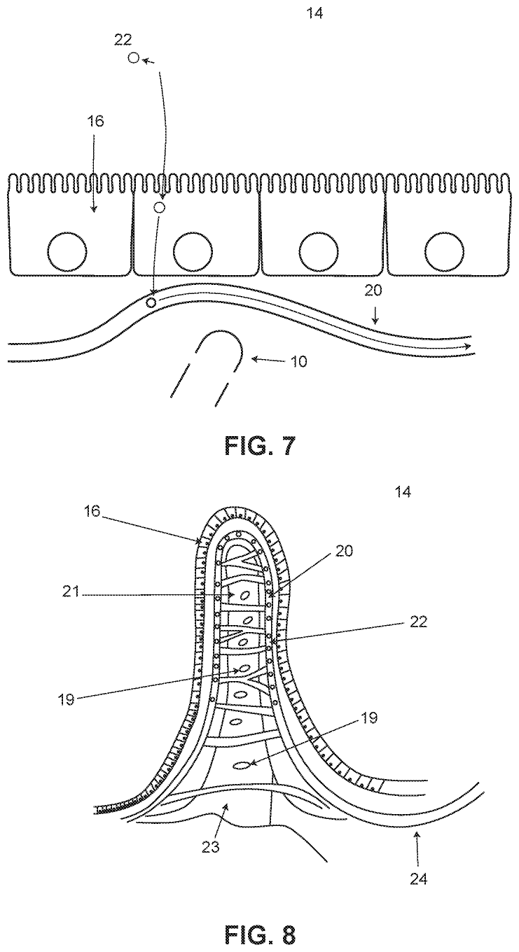

FIG. 7 is an illustration that depicts the route the short-chain and medium-chain fatty acids found within the present invention take when they are metabolized through the human body. The unique fat makeup of the shelf-stable composition (4) results in increased bioavailability of cannabinoids upon digestion. In contrast to FIG. 6, the short-chain fatty acids and medium-chain fatty acids containing cannabinoids (22) travel through the lumen of the intestine (14) without the aid of bile acids and lipases. The short-chain fatty acids and medium-chain fatty acids travel through the epithelial cells of the small intestine (16) without needing to be broken down. They are then transported to the capillary (20) where they make their way to the hepatic portal vein.

FIG. 8 is an illustration that further depicts the different routes the fatty acids contained within the shelf-stable composition are metabolized through resulting in increased bioavailability. Short-chain fatty acids and medium-chain fatty acids are transported by portal bloodstream to the liver. The short-chain fatty acids and medium-chain fatty acids containing cannabinoids travel from the lumen (14) through the epithelial cell and route through the capillary bed (22) where they are transported through the hepatic portal vein (24) as free acids. Short-chain fatty acids and medium-chain fatty acids are more rapidly digested and quickly absorbed in the intestinal lumen (14) and are not stored in the adipose tissue as compared to long-chain fatty acids.

In contrast, chylomicrons containing long-chain fatty acids (19) are too big to enter the capillary bed (20). The chylomicrons are absorbed into the lacteal (21) where they route through the lymphatic vessels and empty through the thoracic duct (23) into the subclavian vein of the circulatory system. Once in the bloodstream, the lipoprotein lipase breaks down the triglycerides of the chylomicrons into free fatty acids and glycerols. These broken down products may now pass through capillary walls where they may be used for energy by cells or stored in adipose tissue as fat. Coconut Oil (2) and Clarified Butter (3) are the preferred oils because they contain short chain fatty-acids, medium chain fatty-acids, and long chain fatty-acids which when digested through the human body results in an increased bioavailability of hydrophobic plant compounds.

Different features, variations and multiple different embodiments have been shown and described with various details. What has been described in this application at times in terms of specific embodiments is done for illustrative purposes only and without the intent to limit or suggest that what has been conceived is only one particular embodiment or specific embodiments. It is to he understood that this disclosure is not limited to any single specific embodiments or enumerated variations. Many modifications, variations and other embodiments will come to mind of those skilled in the art, and which are intended to be and are in fact covered by both this disclosure. It is indeed intended that the scope of this disclosure should be determined by a proper legal interpretation and construction of the disclosure, including equivalents, as understood by those of skill in the art relying upon the complete disclosure present at the time of filing.

What is claimed is:

1. A shelf-stable composition for the infusion of lipophilic plant compounds into food, wherein the shelf-stable composition comprises two or more oils rich in saturated fat and optionally an emulsifier; wherein the two or more oils comprise a mixture of short-chain, medium-chain, and long-chain fatty acids, and wherein the combination of oils increases the bioavailability of lipophilic plant compounds upon consumption.

2. The composition of claim 1, wherein the one or more oils are present in a 1:1 (v/v) ratio.

3. The composition of claim 2, wherein the two or more oils are selected from coconut oil, clarified butter, safflower oil, canola oil, flaxseed oil, sunflower oil, margarine, butter, corn oil, olive oil, sesame oil, soybean oil, peanut oil, cottonseed oil, vegetable shortening, chicken fat, lard, beef tallow, palm oil, cocoa butter, palm kernel oil, rapeseed oil, medium-chain triglycerides oil, avocado oil, hemp oil, milk fat, palm oil, grapeseed oil, mustard oil, rice bran oil, and almond oil.

4. The composition of claim 3, wherein the two or more oils are coconut oil and clarified butter.

5. The composition of claim 1, wherein the emulsifier is selected from lecithin, agar, albumin, alginates, casein, egg yolk, glycerol monostearate, a gum, Irish moss, and soap.

6. The composition of claim 6, wherein the emulsifier is lecithin.

7. The composition of claim 1, wherein the ratio of two or more oils to emulsifier is 16:1 (v/v).

8. A method of making the shelf-stable composition of claim 1 comprising mixing the two or more oils and optionally an emulsifier until evenly blended.

9. The method of claim 8 further comprising packaging the evenly blended shelf-stable composition in an airtight container.

10. The method of claim 9 further comprising storing the packaged composition at room temperature.

11. A method for preparing a cannabinoid infusion comprising heating a mixture of decarboxylated cannabis and a shelf-stable composition of claim 1.

12. The method of claim 11, wherein the mixture is heated for at least 15 minutes.

13. The method of claim 12 wherein the heat is sufficient to simmer the mixture but not to boil the mixture.

14. The method of claim 13, further comprising removing the decarboxylated cannabis from the mixture.

15. A method for improving the oral bioavailability of cannabis, the method comprising heating a mixture of decarboxylated cannabis and a shelf-stable composition of claim 1 for at least 15 minutes at a heat sufficient to simmer but not boil the mixture, wherein the mixture has improved bioavailability compared to a that of a similarly heated mixture of decarboxylated cannabis and butter.

16. The method of claim 15, further comprising removing the decarboxylated cannabis from the mixture of decarboxylated cannabis and a shelf-stable composition of claim 1 after heating and adding the mixture to a food product.

17. A cannabinoid infusion product comprising the shelf-stable composition of claim 1 and one or more lipophilic cannabinoid compounds.
